(19) United States
(12) Patent Application Publication
(10) Pub. No.: US 2011/0200666 A1
(43) Pub. Date: Aug. 18, 2011

(54) UV PROTECTION AGENT FOR THE SKIN OR HAIR HAVING A UV FILTER SUBSTANCE ENCAPSULATED IN A VESICULAR CARRIER SYSTEMü

(75) Inventors: Dirk Teichmüller, Linsengericht (DE); Monika Beyer, Nidderau-Ostheim (DE); Michael Sacher, Schiuchtern (DE)

(73) Assignee: ROVI Cosmetics International GmbH, Schluchtern (DE)

(21) Appl. No.: 12/833,155

(22) Filed: Jul. 9, 2010

(30) Foreign Application Priority Data
Feb. 12, 2010 (DE) 10 2010 001 905.4

(51) Int. Cl. A61K 9/127; A61K 8/73; A61Q 17/04

(52) U.S. Cl. 424/450; 424/59; 424/60; 977/773; 977/907

(57) ABSTRACT

To provide a UV protection agent for topical application to the skin or hair with a very high UV filter substance concentration and good adhesion to skin and hair in accordance with the invention there is proposed a UV protection agent having at least one UV filter substrate encapsulated in a vesicle carrier system, wherein the UV protection agent is characterised in that the at least one UV filter substance encapsulated in the vesicular carrier system is soluble in oil and the vesicular carrier system comprises vesicles which are made up of hydrophobised polysaccharides and are of a particle size of between 10 and 1000 nm and have a positive surface charge with a zeta potential in the range of between 1 and 150 mV. In addition there is proposed the use of such a UV protection agent in suitable cosmetic and/or pharmaceutical formulations.

The present invention concerns a UV protection agent for topical application to the skin or hair having at least one UV filter substance encapsulated in a vesicular carrier system, and the use of such a UV protection agent in corresponding cosmetic and/or pharmaceutical formulations

Sunlight which is visible to the human eye extends over a radiation spectrum of between 400 and 800 nm. Below 400 nm is the spectrum of UV radiation (UV=ultraviolet). Although ultraviolet radiation represents the lowest-energy ionising radiation it can be harmful to surfaces of the body that are exposed to that radiation.

On the skin, UV-A rays (between 320 and 400 nm) cause inter alia damage to collagens, whereby the skin loses elasticity and therefore can prematurely age. In addition increased irradiation in the UV-A range leads to a higher risk of melanomas. UV-B rays (between 280 and 320) lead to inflammation-induced skin reddening and cause sunburn which is known everywhere.

In regard to hair the UV radiation involves a photochemically induced loss of proteins and breakdown of the hair pigment melanin. In that respect UV-B rays involve in particular morphological damage to the hair such as for example breakdown of hair proteins while UV-A rays can cause in particular biochemical changes and changes in color. Amino acids from which the hair proteins are composed are light-sensitive. Their photochemical damage produces free radicals which in turn further damage the protein structure of the keratin and thus the hair.

There is therefore a need for UV protection agents for protection against the above-mentioned harmful effects of the UV component in sunlight. Such UV protection agents are usually applied to the skin or hair to prevent the adverse effects of solar radiation (such as sunburn, skin ageing, damage to the hair). The specified UV protection agents contain suitable UV filter substances for that purpose.

A distinction is made in regard to the UV filter substances between chemical and physical UV filter substances. Chemical filter substances absorb energy-rich radiation and give it off again as lower-energy, longer-wave radiation or heat. Physical filter substances in contrast primarily scatter and reflect the light. As the individual substances generally do not afford any protection over the entire UV spectrum, in general a plurality of substances are combined.

To achieve optimum UV protection there must be a level of UV filter substance concentration that is as high as possible in a UV protection agent. That is only limitedly satisfactorily guaranteed to the desired extent in many conventional UV protection agents. In particular the levels of concentration desired for optimum UV protection are frequently not reached when a combination of two or more different UV substances with a synergistic action spectrum is required in a respectively high level of concentration in order to provide a UV protection agent with optimum wideband protection over various ranges of the relevant UV spectrum.

Therefore the object of the present invention is to provide a UV protection agent in which UV filter substance can be contained in very high levels of concentration. In particular the invention seeks to make it possible to stably introduce two or more different UV filter substances with a synergistic action spectrum in a respective very high level of concentration into the UV protection agent to provide a UV protection agent with an optimum wideband protection over various ranges of the relevant UV spectrum. In addition the invention seeks to provide the possibility of stably integrating into the UV protection agent substances which are capable of protecting UV filter substances from being decomposed by UV radiation and thus stabilising them, referred to as photostabilisers, together with the UV filter substances.

In addition it is desirable for the UV protection agent, with the aforementioned capabilities, also to afford good adhesion to skin and hair in order in that way to ensure a residence time which is as long as possible for the UV filter substance or substances on the skin or hair. In particular the adhesion is to be so effective that the UV filter substance or substances can survive even a plurality of washing processes on the skin or hair.

According to the invention that object is attained by a UV protection agent for topical application to the skin or hair having at least one UV filter substance encapsulated in a vesicular carrier system, wherein the UV protection agent is characterised in that the at least one UV filter substance encapsulated in the vesicular carrier system is soluble in oil and the vesicular carrier system comprises vesicles which are made up of hydrophobised polysaccharides and are of a particle size of between 10 and 1000 nm and have a positive surface charge with a zeta potential in the range of between 1 ands 150 mV.

It was possible to show that the UV protection agents according to the invention afford the advantage that large amounts of oil-soluble UV filter substance can be introduced in stable fashion into a nano-scale, novel carrier system and thus optimise the action of the UV protection agent.

Encapsulation in a positively charged carrier system means that on the one hand the adhesion to skin and hair and thus the residence time of the UV filter substance can be significantly increased. In that respect the preparation according to the invention demonstrably exhibits good film formation on skin and hair, and effective adhesion which survives a plurality of washing processes. Both the amount of UV filter substance remaining on the skin and hair after a number of washing operations and also the protective action thereof could be considerably increased by the preparation according to the invention, in relation to free UV filter substances.

On the other hand encapsulation of the UV filter substance permits incorporation in a high effective level of concentration into hydrophilic cosmetic and/or pharmaceutical formulations. The combination of a plurality of oil-soluble UV filter substances with a synergistic action spectrum in a carrier system is also possible by a UV protection agent having the properties according to the invention. In that way it is possible to achieve extremely effective wideband protection over the UVA and UVB ranges.

The term ‘encapsulation’ in connection with the present invention includes embedding in principle of UV filter substances between the polysaccharide molecules and the nanostructures formed by the polysaccharide molecules as well as the inclusion of the UV filter substances in the vesicle interior.

The lipid vesicles according to the invention have a zeta potential in the range of between 1 and 150 mV. The term ‘zeta potential’ describes the electrical potential of a shearing layer of a moved particle in a suspension. Measurement of the zeta potential can be effected by moving particles through an applied electrical field. The zeta potential can then be calculated from the resulting speed of the particles. Preferred lipid vesicles have a zeta potential of between 30 and 100 mV. In the case of particularly preferred lipid vesicles the zeta potential is between 40 and 60 mV

The improved adhesion of the UV protection agent is based inter alia on the positive charge of the vesicle surface. The positive charge of the vesicle surface leads to improved adhesion of the vesicles to the surface of skin cells. In addition the positive charge of the vesicles also leads to improved adhesion to the hair surface. It was also found that that improved adhesion is also related to the particle sizes of the carrier vesicles, which are nano-scale in accordance with the invention, in which respect however all those findings are not intended to be binding in relation to the present invention and are therefore not intended to restrict the scope of the invention.

The particle size of the lipid vesicles according to the invention is in the range of between 10 and 1000 nm In given embodiments the particle size is between 100 and 400 nm. In other embodiments it is between 100 and 350 nm. In preferred embodiments the particle size is between 100 and 250 nm.

The term ‘particle size’ in connection with the present invention signifies the mean particle size. The mean particle size can be determined by means of photon correlation spectroscopy. Photon correlation spectroscopy—also referred to as dynamic light scattering—is an optical measurement method for determining the size distribution of vesicles and particles in fluids. The method utilises the scattering of laser light by the vesicles.

The combination according to the invention of the positive charge of the vesicle surface and the nano-scale particle size of the carrier vesicles, both in regard to skin and also hair applications, provides that UV filter substance or substances contained in the vesicles remain more durably on the surfaces to be protected.

Preferably the positive charge on the vesicle surface is afforded by the vesicles having positively charged molecules as charge generators, in addition to the polysaccharides from which the vesicles are made up, wherein those charge generators are selected from primary, secondary, tertiary and quaternary alkyl ammonium salts or combinations thereof. Particularly preferred in that respect are C₁₂-C₂₂ alkyl ammonium salts.

Quite particularly preferably the charge generators in the present invention are quaternary C₁₂-C₂₂ alkyl trimethyl ammonium salts (or fatty acid trimonium salts) of the following formula:

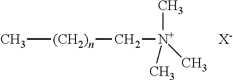

wherein n is an integer of between 10 and 22, and X⁻ is an inorganic or organic anion. Preferably n in the above alkyl trimethyl ammonium salt formula is equal to 22.

Preferably X⁻ in the foregoing formula is a halide ion or the anion of an organic acid which is selected from a cosmetically or pharmaceutically compatible carboxylic acid or sulfonic acid. Particularly preferably X⁻ is bromide, chloride, fluoride, iodide, saccharinate, tosylate or methosulfate.

Preferably the charge generators in the present invention are selected from one or more of ceteartrimonium chloride, cetrimonium bromide, cetrimonium chloride, cetrimonium saccharinate, cetrimonium tosylate, cocotrimonium chloride, cocotrimonium methosulfate, lautrimonium bromide, lautrimonium chloride, myrtrimonium bromide, octyldecyl trimonium chloride, steartrimonium bromide, steartrimonium chloride, steartrimonium methosulfate and steartrimonium saccharinate. In a particularly preferred embodiment of this invention behentrimonium chloride is used as the charge generator.

Preferably in the present invention one or more of the aforementioned positively charged charge generators is so used that the charge generator proportion in the vesicles with respect to the total weight of the vesicles overall is in the range of between 0.01 and 10% by weight. In given embodiments the charge generator proportion is in the range of between 0.01 and 2.0% by weight.

The proportions specified in percent by weight relate in principle to the complete formulation of the UV protection agent in accordance with the composition components, inclusive of the proportion of water required for vesicle formation, unless otherwise specified.

The specified proportion also includes all substances clearly falling within the definition of the respective group of substances. Accordingly the specified proportion of charge generators in the embodiments with a charge generator includes the proportion of that one charge generator and in embodiments with a plurality of charge generators the total of all charge generators. That applies moreover in the same manner to the proportions specified in connection with this invention of hydrophobised polysaccharide, filter substance and adjuvants or other proportions of substance or material.

The basic polysaccharide structure of the hydrophobised polysaccharides which can be used in the UV protection agent for the formation of the vesicles can be selected from all cosmetically or pharmaceutically compatible polysaccharides which are capable of forming vesicles. Preferably this involves water-soluble polysaccharides and/or ethers thereof with short-chain alcohols (C₁ through C₄), wherein the water-soluble polysaccharides can be linear, branched, comb-like and/or stellate. Linear water-soluble polysaccharides are particularly preferred. Copolymers or block copolymers with different monosaccharide units and/or monosaccharide units which are linked together in different ways are also considered.

The hydrophobised polysaccharides from which the vesicles are made up preferably have a basic polysaccharide structure of polyglucose or polyfructose. Preferred hydrophobised polysaccharides with a basic polysaccharide structure comprising a polyglucose are cellulose, methylcellulose, hydroxyethylcellulose, amylose, amylopectin and dextrin. A preferred hydrophobised polysaccharide with a basic polysaccharide structure of polyfructose is inulin.

The hydrophobised polysaccharide in the present invention preferably includes on average between 5 and 1000 monosaccharide units. Still more preferred are between 10 and 500 monosaccharide units. Particularly preferred are between 20 and 100 monosaccharide units.

In special embodiments it is also possible to use mixtures of the aforementioned polysaccharides, with the proviso that those mixtures are capable of forming vesicles.

The proportion of polysaccharides in the vesicles, in relation to the total weight of the vesicles, is in the range of between 1 and 85% by weight. Preferably the proportion of polysaccharide with respect to the total weight of the vesicles is between 5 and 25% by weight. Particularly preferably the proportion of polysaccharide is between 8 and 15% by weight.

In preferred embodiments the hydrophobised polysaccharides from which the vesicles are made up are hydrophobised by having a basic polysaccharide structure with C₃₋₂₂ alkyl groups which are bonded to the hydroxy groups of the polysaccharide by way of alkyl ether bonds or alkyl urethane bonds or which are bonded to the basic polysaccharide structure by, way of a linker (for example polyether linker or polyethylene glycol linker).

The molecular weight of the hydrophobically modified polysaccharide in preferred embodiments is in the range of between 5000 and 500,000 g/mol. Preferably in that respect the range is between 5000 and 100,000 g/mol.

In preferred hydrophobically modified polysaccharides the quotient of the number of hydrophobically modified groups and modifiable groups (degree of modification) is between 0.01 and 0.9. In particularly preferred embodiments the degree of modification is between 0.03 and 0.15.

The hydrophobic groups and also the polysaccharide spine in certain embodiments can be substituted one or more times by for example halogen, hydroxy, alkoxy, amino, alkylamino, aryl, arylalkyl, carboxy, carboxyester and cycloaliphatic residues. Preferably however the hydrophobically modified polysaccharides involve non-ionic compounds.

Hydrophobically modified polysaccharides which are particularly preferably used according to the invention are inulin lauryl carbamate, cetylhydroxyethyl cellulose, hydroxy-C3-6 alkyl-modified cellulose, in particular hydroxypropyl cellulose and hydroxypropyl methylcellulose.

In the present invention all substances which have a chemical or physical UV-filtering action are unrestrictedly considered as the UV filter substance, assuming that they are oil-soluble. The chemical filter substances absorb energy-rich radiation and give it off again as lower-energy, longer-wave radiation or heat. The physical filter substances primarily scatter and reflect the light.

Preferred examples of UV filter substances which can be employed in the present invention are without limitation thereto 3-benzylidene camphor, 4-methylbenzylidene camphor, benzophenone-1, benzophenone-2, benzophenone-3, benzophenone-4, benzophenone-5, benzophenone-6, benzophenone-9, benzylidene camphor sulfonic acid, bis-ethylhexyloxyphenol methoxyphenyline triazine, butylmethoxydibenzoyl methane, camphor benzalkoniummethosulfate, diethylaminohydroxybenzoylhexylbenzoate, diethylhexylbutamidotriazone, dinatriumphenyldibenzimidazole tetrasulfonate, drometrizole trisiloxane, ethylhexyldimethylpaba, ethylhexylmethoxycinnamate, ethylhexylsalicylate, ethylhexyltriazone, homosalate, isoamyl-p-methoxycinnamate, methylene-bis-benzotriazolyl tetramethylbutylphenol, octocrylene, PEG-25-paba, phenylbenzimidazole sulfonic acid, polyacrylamidomethylbenzilidene camphor, polysilicone-15, potassium phenylbenzimidazole sulfonate, sodium mangoseedate, sodium phenylbenzimidazole sulfonate, tea-phenylbenzimidazole sulfonate, terephthalydene dicamphor sulfonic acid, ferulic acid, cinoxate, diisopropylmethyl cinnamate, 4-(2-beta-glucopyranosiloxy)propoxy-2-hydroxybenzophenone, glycerylethylhexanoate dimethoxycinnamate, and isopentyltrhnethoxycinnamate trisoloxane.

Preferably the proportion of UV filter substance in the present invention is between 1 and 65% by weight with respect to the total weight of the complete formulation of the UV protection agent in accordance with the composition components, inclusive of the proportion of water required for vesicle formation.

As the individual filter substances generally do not provide any protection over the entire UV spectrum, preferred embodiments of the invention provide for combining a plurality of filter substances to achieve a UV protection which is as wide as possible.

According to the invention the UV protection agent is used in cosmetic and/or pharmaceutical formulations, wherein pharmaceutical formulations are those which are covered by the law governing the manufacture and prescription of drugs.

The formulations can include all adjuvant and additive substances which are usually employed in relation to cosmetic or pharmaceutical preparations. In particular the term ‘adjuvant substance’ in connection with the present invention embraces such additive substances which act on the physical properties of the vesicles and the stability thereof and/or serve for preservation of the UV protection agents. Examples of such adjuvant substances are oils, alcohols, polyols, anti-oxidants, gel-forming agents, buffers, preserving agents, bactericides and germ inhibitors, consistency additives, complexing agents and thickeners.

Preferably the UV protection agent according to the invention is characterised in that the proportion of adjuvant with respect to the total weight of the complete formulation of the UV protection agent in accordance with the composition components, inclusive of the proportion of water required for vesicle formation, is in the range of between 0.01 and 10% by weight.

Preferably the UV protection agent according to the invention is used for cosmetic and/or pharmaceutical formulations which are suited to topical application. The UV protection agent according to the invention can be present for that purpose in all formulations suitable for topical application, for example in the form of a gel, a cream, an ointment, a spray or a lotion. For that purpose the UV protection agent according to the invention can be incorporated into a carrier matrix. The carrier matrix may involve gel formulations, cream formulations, lotions, mask applications and so forth.

For uses in the skin area, the UV protection agent according to the invention is preferably used in a lotion, a cream, an ointment, a gel, an aqueous fluid, a face lotion, a sun product or a mask.

For uses in the hair area, the UV protection agent according to the invention is preferably used in a shampoo (preferably with mild surfactants), a rinsing agent, a hair gel, a conditioner, a hair tonic, a hair styling product or a hair care product.

It will be appreciated that all components of the UV protection agent and its formulations according to the invention involve pharmaceutically, cosmetically or dermatologically compatible substances. In accordance with this invention a substance is pharmaceutically, cosmetically or dermatologically compatible if it is non-toxic and can be topically applied in the case of the majority of potential users without the user spontaneously or after a while suffering from an unwanted physiological reaction such as for example reddening or the occurrence of itching.

For the purposes of the original disclosure it is pointed out that all features as can be seen by a man skilled in the art from the present description and the claims, even if they are described in specific terms only in connection with certain other features, can be combined both individually and also in any combinations with others of the features or groups of features disclosed here insofar as that has not been expressly excluded or chemical, physical-chemical, cosmetic, pharmacological or dermatological aspects make such combinations impossible or meaningless. A comprehensive explicit representation of all conceivable combinations of features is dispensed with here only for the sake of brevity and readability of the description.

1. A UV protection agent for topical application to the skin or hair having at least one UV filter substance encapsulated in a vesicular carrier system, wherein the at least one UV filter substance is soluble in oil and the vesicular carrier system comprises vesicles which are made up of hydrophobised polysaccharides and are of a particle size of between 10 and 1000 nm and have a positive surface charge with a zeta potential in the range of between 1 and 150 mV.

2. A UV protection agent as set forth in claim 1 wherein the proportion of UV filter substance in the vesicles with respect to the total weight of the UV protection agent is in the range of between 1 and 65% by weight.

3. A UV protection agent as set forth in claim 2 wherein the proportion of polysaccharides in the vesicles with respect to the total weight of the vesicles is in the range of between 1 and 85% by weight.

4. A UV protection agent as set forth in claim 1 wherein the positive surface charge is produced by the vesicles in addition to the polysaccharides from which the vesicles are built up having positively charged molecules as charge generators, said charge generators being selected from primary, secondary, tertiary and quaternary alkyl ammonium salts or combinations thereof.

5. A UV protection agent as set forth in claim 4 wherein the proportion of charge generators in the vesicles with respect to the total weight of the UV protection agent is in the range of between 0.01 and 10% by weight.

6. A UV protection agent as set forth in claim 1 the hydrophobised polysaccharides from which the vesicles are made up have a basic polysaccharide structure of polyglucose or polyfructose.

7. A UV protection agent as set forth in claim 1 wherein the hydrophobised polysaccharides from which the vesicles are made have a basic polysaccharide structure comprising a polyglucose selected from cellulose, methylcellulose, hydroxyethylcellulose, amylose, amylopectin or dextrin, or a basic polysaccharide structure comprising the polyfructose inulin.

8. A UV protection agent as set forth in claim 1 wherein the hydrophobised polysaccharides from which the vesicles are made have a basic polysaccharide structure hydrophobically modified by C₃₋₂₂ alkyl groups which are bonded to the hydroxy groups of the polysaccharide by way of alkyl ether bonds or by way of alkyl urethane bonds or are bonded to the basic polysaccharide structure by way of a linker.

9. A UV protection agent as set forth in claim 1 wherein the at least one UV filter substance has a chemical or physical UV filter action.

10. A UV protection agent as set forth in claim 1 wherein the at least one UV filter substance is selected from 3-benzylidene camphor, 4-methylbenzylidene camphor, benzophenone-1, benzophenone-2, benzophenone-3, benzophenone-4, benzophenone-5, benzophenone-6, benzophenone-9, benzylidene camphor sulfonic acid, bis-ethylhexyloxyphenol methoxyphenyline triazine, butylmethoxydibenzoyl methane, camphor benzalkoniummethosulfate, diethylaminohydroxybenzoylhexylbenzoate, diethylhexylbutamidotriazone, dinatriumphenyldibenzimidazole tetrasulfonate, drometrizole trisiloxane, ethylhexyldimethylpaba, ethylhexylmethoxycinnamate, ethylhexylsalicylate, ethylhexyltriazone, homosalate, isoamyl-p-methoxycinnamate, methylene-bis-benzotriazolyl tetramethylbutylphenol, octocrylene, PEG-25-paba, phenylbenzimidazole sulfonic acid, polyacrylamidomethylbenzilidene camphor, polysilicone-15, potassium phenylbenzimidazole sulfonate, sodium mangoseedate, sodium phenylbenzimidazole sulfonate, tea-phenylbenzimidazole sulfonate, terephthalydene dicamphor sulfonic acid, ferulic acid, cinoxate, diisopropylmethyl cinnamate, 4-(2-beta-glucopyranosiloxy)propoxy-2-hydroxybenzophenone, glycerylethylhexanoate dimethoxycinnamate, and isopentyltrimethoxycinnamate trisoloxane.

11. A UV protection agent as set forth in claim 1 including one or more adjuvants.

12. A UV protection agent as set forth in claim 11 wherein the proportion of adjuvant in the vesicles in relation to the total weight of the UV protection agent is in the range of between 0.01 and 10% by weight.

13. Use of a UV protection agent as set forth in claim 1 for the production of a cosmetic and/or pharmaceutical formulation.

14. Cosmetic and/or pharmaceutical formulation including a UV protection agent as set forth in claim 1.
